(19) United States
(12) Patent Application Publication
(10) Pub. No.: US 2014/0271729 A1
(43) Pub. Date: Sep. 18, 2014

(54) USE OF BIOGENIC AMINES SPERMINE AND SPERMIDINE FOR THE HOMEOPATHIC TREATMENT OF HORMONE IMBALANCES

(71) Applicant: APOTHECA, INC., Woodbine, IA (US)

(72) Inventor: Jesse Rettig, Woodbine, IA (US)

(73) Assignee: Apotheca, Inc., Woodbine, IA (US)

(21) Appl. No.: 13/835,415

(22) Filed: Mar. 15, 2013

(51) Int. Cl. A61K 31/132; A61K 45/06

(52) U.S. Cl. CPC A61K 31/132; A61K 45/06
USPC 424/400; 514/674; 424/623; 424/725; 424/730; 564/512

(57) ABSTRACT

This invention relates to a composition, and methods of administration and treatment comprising a homeopathic preparation of an effective amount of Spermine and/or Spermidine that improves hormone imbalances in males and females. The purpose of the invention is to provide a homeopathic treatment comprising Spermine and/or Spermidine that demonstrates improvement in energy, sleep, sexual function, relief of prostrate symptoms, lighter and shorter hot flush symptoms, stamina, weight loss, and the overall mood and stress of a patient.

### FIELD OF THE INVENTION

This invention relates to a composition, and methods of administration and treatment comprising a homeopathic preparation of an effective amount of Spermine and/or Spermidine that improves hormone imbalances in males and females.

### BACKGROUND OF THE INVENTION

Homeopathy, which dates back to the nineteenth century, is founded on the principles of pharmacology and biology. In 1877, Hugo Schultz postulated that the effect of a stimulus on a living cell is indirect and proportional to its intensity and quantity. Later, in 1888, Schultz demonstrated that very low concentrations of yeast toxins increased yeast growth over 100 fold. Concurrently, the psychiatrist Rudolph Arndt developed his “Basic Law of Biology,” which states that weak stimuli slightly accelerate the vital activity, middle-strong stimuli raise it, strong stimuli suppresses it, and very strong stimuli halt vital activity. These separate observations were formulated by Arndt in 1888 into one of the earliest laws of pharmacology representing the homeopathic effect, the Arndt-Schultz law, which states: every stimulus on a living cell elicits an activity, which is inversely proportional to the intensity of the stimulus (Martius F. Das Arndt-Schultz Gnindgesetz, Muench Med. Wschr., 1923, 70(31):1005-1006). This law was later restated by Hueppe as: for every substance, small doses stimulate, moderate doses inhibit, large doses kill. Allopathic medicine, with its emphasis on moderate drug doses, works to inhibit undesired physical symptoms and to kill undesired pathogens. Homeopathic medicine begins with small doses and moves towards higher and higher dilutions to stimulate the body's own natural electromagnetic forces.

One of the basic tenets of homeopathic medicine is that a cure for a disease can be evoked by using a high dilution medicine that resembles but is different from the cause of the disease. Homeopathy is widely accepted as a useful therapeutic throughout Europe, the British Commonwealth countries and India, and has been demonstrated to have characteristic and reproducible effects. A critical review of more than 100 controlled and/or clinical studies of homeopathy determined that patients received positive healing benefits from homeopathy beyond the placebo effect (Kleijnen, J. et al. 1991 Brit. Med. J. 302:316-323; Linde, K., Clausius, N., Ramirez, G., Melchart, D., Eitel, F., Hedges, L. V., Jonas, W. B., 1997, Lancet, 350:834-843; Reilly, D., et al, 1994, Lancet, 344:1601-1608).

A common principle of homeopathy is the Law of Similars, which was founded in the science of pharmacology and states that a drug has two effects on the body, a direct effect and the subsequent reaction of the body to the drug, evoking symptoms or side effects. In homeopathy, as the drug is diluted, some of the positive benefits of the drug remain, plus new characteristics of the drug become available to the body which not only alleviate side effects, but have new characteristic features that actually ameliorate other symptoms the person may have.

While the exact mechanism of action of homeopathic medicines is unknown, magnetic resonance image measurements on serial dilutions of substances indicate that the hydroxyl (OH) groups in the solvent of solutions continue to change as dilutions become successively higher (Sacks, A. D. 1983 J. Holistic Med. 5:175-176; Smith, R. and Boericke, G. 1968 J. Am. Inst. Homeopathy 61:197-212; Smith, R. and Boericke, G. 1966 J. Am. Inst. Homeopathy 59:263-279). It is clear that the specific effects of homeopathics are of a non-molecular origin, yet provide potent biological activities that are clinically effective. It has been postulated that highly dilute compounds transfer biological activity to cells by electromagnetic fields (Benveniste, J. 1993 Frontier Perspectives 3:13-15). Del Giudice et al. have hypothesized that interactions between the electric dipoles of water and the radiation fields of a charged molecule generate a permanent polarization of water which becomes coherent and has the ability to transmit specific information to cell receptors, somewhat like a laser (Del Giudice, E., Preparata, G., Vitiello, G. 1988, Phys. Rev. Lett. 61:1085-1088).

The drug involved with the present homeopathic invention originated from the use of radish leaves as a medicine. There are a number of patent documents and non-patent documents which teach the use of radish leaves to treat various ailments. Indeed it was generally known that radishes and radish leaves had a beneficial effect on health in humans. For example, U.S. Pat. No. 4,059,695 describes a type of tea that has radish leaves as one of its ingredients which acts as a general tonic for humans. As well, U.S. Pat. No. 5,498,412 describes a fermented mixture of various things including radish leaves which act as a natural antioxidant. As well, in the popular literature, a publication from 1982 describes the use of black radish in medicine (Slutomski, et al. “Black radish as a source of various phyto pharmaceuticals”) (1982). Another paper describes the inhibition of mutagenicity through use of radish extract (Badrid, S. A. (1994) Cancer Letters 84:1-5). In addition, in the Encyclopedia of Herbal Medicine, radish is listed as having various health keeping properties.

A chemical analysis has been performed in respect of radishes namely the chemical constituents of raphanus sativus L (Brassicaceae) (The Clinicians' Handbook of Natural Healing by Gary Null, Kensington Publishing, New York, 1997). Over 90 different chemicals and compounds are identified in the list of constituents, including Spermine and Spermidine. Therefore, according to embodiments of the present invention, the inventor has identified Spermine and Spermidine as the active constituents capable of having a strong effect on our body cells through homeopathic preparations and administration, including those organs and glands that affect hormone production. According to aspects of the present invention, through the use of controlled homeopathic preparation and administration, the present invention has a balancing effect with regard to hormone imbalances in males and females, and demonstrates improvement in energy, sleep, sexual function, fertility, libido, relief of prostrate symptoms, lighter and shorter hot flush symptoms, stamina, weight loss, and the overall mood and stress of a patient.

According to one aspect of the present invention, a homeopathic composition that improves hormone imbalances in males and females is provided.

According to another aspect of the present invention, a method of treating hormone imbalances, disorders and conditions by administering homeopathic preparations comprising Spermine and/or Spermidine, is provided.

According to a further aspect of the present invention a method of administering homeopathic preparations comprising Spermine and/or Spermidine for treating hormone imbalances, disorders and conditions in males and females, is provided.

Different aspects may meet different objects of the invention. Other objectives and advantages of this invention will be more apparent in the following detailed description taken in conjunction with the figures. The present invention is not to be limited by or to these objects or aspects.

### BRIEF SUMMARY OF THE INVENTION

Therefore, it is a principal object, feature, and/or advantage of the present invention to overcome the aforementioned deficiencies in the art of medicine and provide a composition comprising a homeopathic preparation of an effective amount of Spermine and/or Spermidine that improves hormone imbalances in males and females.

Another object, feature, and/or advantage of the present invention is to provide a method for treating hormone imbalances, disorders and conditions by administering a composition comprising a homeopathic preparation of an effective amount of Spermine and/or Spermidine.

A further object, feature, and/or advantage of the present invention is to provide a method of administering a composition comprising a homeopathic preparation of an effective amount of Spermine and/or Spermidine that improves hormone imbalances in males and females.

Another object, feature, and/or advantage of the present invention is to provide a homeopathic treatment comprising administering homeopathic preparations comprising an effective amount of a composition including Spermine and/or Spermidine that demonstrates improvements in energy, sleep, sexual function, fertility, libido, relief of prostrate symptoms, lighter and shorter hot flush symptoms, stamina, weight loss, and the overall mood and stress of a patient.

A further object, feature, and/or advantage of the present invention is to provide a method for enhancing fertility and libido of a patient using a homeopathic composition.

These and/or other objects, features, and/or advantages of the present invention will be apparent to those skilled in the art. The present invention is not to be limited to or by these objects, features, and advantages. No single aspect need provide each and every object, feature, or advantage.

### DESCRIPTION OF FIGURES

The invention will now be described in relation to the figures in which:

FIG. 1 represents the chemical structures of the polyamine Spermidine and Spermidine's analog and post-cursor, Spermine.

FIG. 2 is a table containing biochemical information about Spermidine.

FIG. 3 is a table showing homeopathic dilution designations as applied according to embodiments of the invention.

Various embodiments of the present invention will be described in detail with reference to the drawings, wherein like reference numerals represent like parts throughout the several views. Reference to various embodiments does not limit the scope of the invention. Figures represented herein are not limitations to the various embodiments according to the invention and are presented for exemplary illustration of the invention.

### DETAILED DESCRIPTION OF THE INVENTION

### Spermidine and Spermine:

Spermidine and its analog and post-cursor, Spermine, are polyamines. See FIG. 1. Polyamines are polycationic compounds, which are found in significant amounts in nearly every prokaryotic and eukaryotic cell type, including microbes. They are formed by the enzymatic decarboxylation of the amino acids ornithine or arginine. Spermine was first identified in 1678, by Van Leewenheuk. He identified crystals in semen that were later identified as the tetraamine Spermine. The triamine Spermidine was identified in the early twentieth century. See FIG. 2.

Spermine is a polyamine involved in cellular metabolism found in all eukaryotic cells. Formed from Spermidine, it is found in a wide variety of organisms and tissues and is an essential growth factor in some bacteria. It is found as a polycation at physiological pH. Spermine is associated with nucleic acids and is thought to stabilize helical structure, in particular, in viruses.

Despite the ubiquitous nature of these compounds, the precise roles that polyamines play in cellular physiology and pathology are still being investigated. However, Spermidine and Spermine are known to bind nucleic acids, stabilize membranes and stimulate the activity of several enzymes such as RNA polymerase. Polyamines have also been investigated as potentially important anti-cancer and anti-infective agents. Polyamine content is increased in many cancers arising from epithelial cells. Other potential areas of research include modulation of the NMDA receptor, anti-diarrheal agents, polyamine based venoms, potential carriers for drug delivery and boron-neutron capture therapy. They also are known to exert their effects in part by regulating gene expression.

Although simple in structure, the polycationic polyamines Spermidine and Spermine and their diamine precursor Putrescine are essential factors for growth in eukaryotic cells. For instance, Spermine and Spermidine are polyamines that are essential for healthy cell development and have a profound effect on RNA. It appears that Spermine and Spermidine are inter-related, as the combined benefits are significantly greater than when separated. When Spermidine and Spermine are absorbed into the blood stream (70% absorption), they have a strong effect on human body cells, including those organs and glands that affect hormone production. The consequence of this has been a balancing effect with improvement in fertility and libido.

Spermine and Spermidine may be the compounds as isolated from green plant materials, such as radish leaves, stems, and seeds, corn, oats, cucumber, lettuce, lentil seeds, and cabbage. As referenced herein, the terms Spermine and Spermidine also include any and all synthetic analogs, derivatives and/or preparations of these naturally occurring polyamines, or biologically active portions thereof, howsoever prepared. One skilled in the art will ascertain the various commercially-available sources of such naturally-extracted and/or synthesized components which are suitable for use in the compositions and methods of the present invention.

Homeopathic Composition that Improves Hormone Imbalances:

According to one aspect of the present invention, a composition comprising a homeopathic preparation of an effective amount of Spermine and/or Spermidine that improves hormone imbalances in males and females (among other benefits disclosed herein), is provided. An “effective amount” as used herein means the amount required at dosages and for periods of time necessary to achieve the specified desired result.

Many homeopathic medicines are used at concentrations of micrograms (10⁻⁶ M) and nanograms (10⁻¹² M); however, in other homeopathic preparations, the dilutions exceed Avogadro's number (6.023×10⁻²³). When homeopathic compounds are diluted by a factor of 10 (i.e. 1:9 ratio of 1 part mother tincture and 9 parts solvent) with repeated successions (similar to vortexing) and repetitively diluted by this procedure at least 24 times, a potency is achieved (10⁻²⁴) that is so highly dilute that the probability of a single molecule of the original substance remaining in the volume used is less than 1×10⁻¹⁰. Such dilutions according to homeopathy provide increasing potency of a compound with increasing dilutions.

In traditional homeopathic practice, the standard homeopathic dosage is 10-15 drops of a 10⁻¹² molar, or 6 C, solution administered two to three times per day. A 10⁻⁶⁰ molar or 30 C may be given one to three times per day. A 10⁻⁴⁰⁰ molar or 200 C may be given only one time per month or year. A 6 C dilution approximates 1 picogram/ml, which is used in cell culture but would be considered a lower than physiological dose when administered to a patient either orally, topically or by injection. A table showing each of the letter designations and their dilution rate as applied according to the present invention is illustrated in FIG. 3.

The homeopathic preparation of the present invention typically comprise Spermidine and/or Spermine present in a dilution of from about 1× to about 100× in a pharmaceutically acceptable diluent. Homeopathic preparations also comprise a dilution from about 1× to about 200 C or less of Spermidine and/or Spermine and, alternatively or additionally, comprise a dilution from about 1× to about 1 LM of Spermidine and/or Spermine or less. Without being limited according to the invention, all ranges recited for the dilution ranges for the actives of the homeopathic preparations are inclusive of the numbers defining the range and include each integer within the defined range.

The compositions according to the present invention are prepared according to standard Hahnemannian principles, wherein one part, comprised of Spermidine and/or Spermine power, may be mixed with nine parts of diluent and successed 10 times to form a 1× potency. For higher dilutions, the Korsakovian method of dilution using a single container for a series of dilutions rather than a new container for each, will be utilized for dilutions up to the 1 LM potency.

The homeopathic preparations according to the invention further include a diluent (or inactive component). In an aspect, pharmaceutical diluents, excipients or carriers are employed. Suitable pharmaceutical diluents, excipients, or carriers are inactive ingredients may be added in addition to the active drug. These additives may be used as binders, disintegrants, or flavor enhancers. Exemplary diluents include sterile water, starch, cellulose derivatives (e.g. dextrose, lactose), and magnesium stearate (a lubricant). In addition to such diluents, other pharmaceutically-acceptable carriers, binders, and/or excipients may be employed according to the skill of those in the art to which the invention pertains. Further description of such components can be found, for example, in the United States Homeopathic Pharmacopoeia.

The homeopathic preparations according to the invention may optionally include additional components. Non-limiting examples that may be used in combination with homeopathic preparations of Spermidine and Spermine include arsenicum, pulsatilla, aconite, hypericum, metabolic sarcodes, vitamins, minerals, amino acids, herbal extractions, and traditional homeopathic preparations of ingredients. The same potencies, or combinations of potencies, of homeopathic preparations of Spermidine and Spermine and additional constituents are used regardless of whether the administration is in liquid, solid, spray, topical, transdermal or injectable form.

The homeopathic preparations according to the invention may be provided in various composition formulations. The preparations may include oral formulations (e.g. liquid or solid forms), sublingual formulations (e.g. liquid or solid forms), intranasal, topical and/or transdermal formulations, and/or injectable formulations. According to a preferred aspect of the invention, the homeopathic preparations are provided in oral and/or sublingual formulations. In a most preferred aspect of the invention, sublingual formulations are provided to administer homeopathic dosages of the Spermidine and/or Spermine having rapid absorption into the blood stream of a patient in need thereof.

Homeopathic preparations of the composition comprising an effective amount of Spermine and/or Spermidine may be prepared in a chewable tablet form. The tablets are made from a suitable organic material, such as lactose, or sucrose by methods well known in homeopathy, as described in the United States Homeopathic Pharmacopoeia. In particular, tablets are generally produced in two forms, as tablet triturates or compressed tablets. Tablet triturates are produced by preparation of an effective amount of Spermine and/or Spermidine, as previously described, and adding binders as necessary. Binding solutions are composed of a binder, such as gum arabic, microcrystalline cellulose, a preservative if necessary, an inert lubricant, and purified water. The tablets are then molded by hand or preferably by automated equipment, and the tablets are then dried by introducing them into a dehumidified environment with a relative humidity of 35-40%, and an ambient temperature of 70 to 110 degrees Fahrenheit. Compressed tablets are formed by compression of a dry material and contain no special coating. They are compressed from powdered or crystalline solids, and, as with tablet triturates, may contain binders, excipients, lubricants, and disintegrators. Compressed tablets are produced by adding or spraying the homeopathic preparation of an effective amount of Spermine and/or Spermidine to the lactose preparation until thoroughly moistened. Binders may be added at this time as necessary, as described above for tablet triturates. The moistened material is granulated by passing through an appropriate mesh screen, and the moistened granulation is introduced into a dehumidified environment and subsequently dried as described above. The dried granulation is then regranulated through the mesh screen and lubricants, such as mineral oil, talc, calcium stearate, corn starch, are added as necessary. The mixture is then compressed in a rotary tablet compressor or any similar apparatus to the desired tablet size.

Homeopathic Treatment that Improves Hormone Imbalances:

According to another aspect of the present invention, a method is provided for treating hormone imbalances, disorders and conditions by administering a composition comprising a homeopathic preparation of an effective amount of Spermine and/or Spermidine. The method comprises providing a patient, male or female, that has hormone imbalances, disorders and conditions the homeopathic preparations according to the invention. The patient may be a mammal for example selected from the group consisting of human, bovine, feline, equine, porcine, canine, camel, and sheep. The hormone imbalances, disorders and conditions may include for example: rapid aging of the skin; fatigue; irritability; pain in the hips and low back; urinary difficulties; sleep deprivation; infertility; depressed libido; impaired sexual function; low Testosterone levels; and feminization in males.

The method further comprises administering to the patient a composition comprising a homeopathic preparation of an effective amount of Spermine and/or Spermidine, as set forth according to the various compositions of the present invention, to improve hormone imbalances, disorders and conditions. The composition comprises Spermidine and/or Spermine present in a dilution of from about 1× to about 100× in a pharmaceutically acceptable diluent. Homeopathic preparations according to the present invention may also comprise a dilution from about 1× to about 200 C or less of Spermidine and/or Spermine and, alternatively or additionally, comprise a dilution from about 1× to about 1 LM of Spermidine and/or Spermine or less.

The method of treatment according to the invention further comprises administering an effective amount of the composition to the patient over a period of time to improve hormone imbalances, disorders and conditions. Administering an effective amount of the composition may include 1-10 doses of the composition on the first day of treatment, preferably 3 doses. A dosage may comprise 1-20 drops of the composition comprising Spermidine and/or Spermine present in a dilution in a pharmaceutically acceptable diluent. Most preferably, a dosage comprises 10 drops of the composition comprising Spermidine and/or Spermine present in a dilution of about 30× in a pharmaceutically acceptable diluent. If no symptoms of improvement or decline are shown after approximately 24 hours, then the dosages may be repeated. Dosages may be continued in this manner until hormone imbalances, disorders and conditions are improved.

Improvements in hormone imbalances, disorders and conditions may comprise: increased energy; increased libido; increased vaginal wetness; relief of prostrate symptoms; improved sleep; increased fertility; lighter and shorter hot flush symptoms; improved stamina; increased sexual energy; drainage and detoxification; immunomodulation; and cell and organ support; improved sexual function; weight loss; decreased anxiety; improvements in headaches; regularity of periods; improved memory retention; stimulated hair growth; reduction in depression; improvement in aching of joints; reduction in acne; and improves the overall mood and stress of a patient. Results of hormone improvements can also be demonstrated by a reduction in Cortisol levels in males and females; increased testosterone levels in males and females; increased Dehydroepiandrosterone (“DHEA”) levels in males and females; a reduction in Estradiol levels in males; increased Estradiol levels in females; increased Progesterone levels in females; and decreased Progesterone levels in males.

The administration of the composition to the patient may be via oral, sublingual, or topical delivery systems, or using eye drops, nasal or throat sprays, transdermal delivery, or other routes of administration that do not involve injection and that do not require sterile equipment or the participation of health care professionals. Alternatively, the present invention may also be administered by means of intracutaneous, intramuscular, intravenous, or subcutaneous injection.

### Administration of Homeopathic Composition for Improving Hormone Imbalances:

According to a further aspect of the present invention a method of administering a composition comprising a homeopathic preparation of an effective amount of Spermine and/or Spermidine that improves hormone imbalances in males and females, is provided. The method comprises providing a composition comprising a homeopathic preparation of an effective amount of Spermine and/or Spermidine to improve hormone imbalances, disorders and conditions. The composition comprises Spermidine and/or Spermine present in a dilution of from about 1× to about 100× in a pharmaceutically acceptable diluent. Homeopathic preparations may also comprise a dilution from about 1× to about 200 C or less of Spermidine and/or Spermine and, alternatively or additionally, comprise a dilution from about 1× to about 1 LM of Spermidine and/or Spermine or less. The composition is prepared according to standard Hahnemannian principles. One gram, comprised of Spermidine and/or Spermine power, may be mixed with nine grams of diluent and successed 10 times to form a 1× potency. A similar process may be repeated for each potency up to the 1 LM potency.

Suitable pharmaceutical diluents, excipients, or carriers are inactive ingredients that may be added in addition to the Spermine and/or Spermidine. These additives may be used as binders, disintegrants, or flavor enhancers. Some very common diluents include sterile water, starch, cellulose derivatives, and magnesium stearate (a lubricant). The preferred homeopathic diluents for oral administration are a solution of purified water, glycerin, citric acid and a preservative such as sodium benzoate; or a solution of purified water, glycerin, and/or potassium sorbate. Other diluents for oral delivery, including various alcohol-containing solutions, are known in the art and may be employed in the present invention to increase solubility and stability of the composition comprising an effective amount of Spermine and/or Spermidine.

The method further comprises administering to a patient an effective amount of the composition to improve hormone imbalances, disorders and conditions. The administration may be via orally, in liquid or solid form, such as pellets or tablets. Administration may also be via sublingual, or topical delivery systems, or using eye drops, nasal or throat sprays, transdermal delivery, or other routes of administration that do not involve injection and that do not require sterile equipment or the participation of health care professionals. Lotions for topical and transdermal application, and buffered salt solutions for eye applications, are well characterized and widely used in the cosmetic industry, which are readily adaptable to the preparation of the present invention. Additionally, carrier solutions for intranasal administration of substances are well known in the art and widely used in drug delivery systems. Alternatively, the present invention may also be administered by means of intracutaneous, intramuscular, intravenous, or subcutaneous injection.

The homeopathic preparation of this invention comprising Spermine and/or Spermidine, as well as methods of treatment of hormone imbalances and administration for delivery, are universally applicable to all patients of any age, health, and weight of the patient, nature and extent of symptoms, kind of concurrent treatment, frequency of treatment, and the effect desired. Although the invention has been described and illustrated with respect to preferred aspects thereof, it is not to be so limited since changes and modifications may be made therein which are within the full intended scope of the invention.

What is claimed is:

1. A composition comprising:
a homeopathic preparation of an effective amount of Spermine and/or Spermidine that improves hormone imbalances, disorders and conditions in a patient.

2. The composition of claim 1, wherein the Spermine and/or Spermidine are present in a dilution of from about 1× to about 100× in a pharmaceutically acceptable diluent.

3. The composition of claim 1, wherein the Spermine and/or Spermidine are present in a dilution of from about 1× to about 200 C in a pharmaceutically acceptable diluent.

4. The composition of claim 1, wherein the Spermine and/or Spermidine are present in a dilution of from about 1× to about 1 LM in a pharmaceutically acceptable diluent.

5. The composition of claim 2, additionally comprising at least one component selected from the group consisting of: arsenicum, pulsatilla, aconite, hypericum, metabolic sarcodes, vitamins, minerals, amino acids, herbal extractions, and traditional homeopathic preparations of ingredients.

6. The composition of claim 2, wherein the improvements in hormone imbalances, disorders and conditions comprise improvements in energy, sleep, sexual function, fertility, libido, relief of prostrate symptoms, lighter and shorter hot flush symptoms, stamina, weight loss, and the overall mood and stress of the patient.

7. The composition of claim 6, wherein the Spermine and/or Spermidine are synthetically derived.

8. The composition of claim 7, wherein the composition is a liquid.

9. The composition of claim 7, wherein the composition is impregnated on a solid medium.

10. A method of treating hormone imbalances, disorders and conditions, comprising:
identifying a patient that has hormone imbalances, disorders and conditions;
providing a composition comprising a homeopathic preparation of an effective amount of Spermine and/or Spermidine; and
administering an effective amount of the composition to the patient to improve the hormone imbalances, disorders and conditions.

11. The method of claim 10, wherein the Spermine and/or Spermidine are present in a dilution of from about 1× to about 100× in a pharmaceutically acceptable diluent.

12. The method of claim 10, wherein the Spermine and/or Spermidine are present in a dilution of from about 1× to about 200 C in a pharmaceutically acceptable diluent.

13. The method of claim 10, wherein the Spermine and/or Spermidine are present in a dilution of from about 1× to about 1 LM in a pharmaceutically acceptable diluent.

14. The method of claim 11, wherein administering an effective amount of the composition comprises three doses of the composition administered on the first day of treatment, wherein a dosage comprises 10 drops of the composition comprising Spermidine and/or Spermine present in a dilution of about 30× in a pharmaceutically acceptable diluent, further wherein the doses are repeated after approximately every 24 hours in this manner until hormone imbalances, disorders and conditions are improved.

15. The method of claim 14, wherein the administration of the composition to the patient comprises oral, sublingual, topical, eye drops, nasal or throat sprays, and/or transdermal delivery.

16. The method of claim 15, wherein the Spermine and/or Spermidine are synthetically derived.

17. A method of administering a homeopathic preparation of Spermine and/or Spermidine to improve hormone imbalances in males and females, comprising:
providing a composition comprising a homeopathic preparation of an effective amount of Spermine and/or Spermidine; and
administering an effective amount of the composition to the patient to improve the hormone imbalances, disorders and conditions; and
wherein the administration of the composition to the patient comprises oral, sublingual, topical, eye drops, nasal or throat sprays, and/or transdermal delivery.

18. The method of claim 17, wherein the Spermine and/or Spermidine are present in a dilution of from about 1× to about 100× in a pharmaceutically acceptable diluent.

19. The method of claim 17, wherein the Spermine and/or Spermidine are present in a dilution of from about 1× to about 200 C in a pharmaceutically acceptable diluent.

20. The method of claim 17, wherein the Spermine and/or Spermidine are present in a dilution of from about 1× to about 1 LM in a pharmaceutically acceptable diluent.
